(19) United States
(12) Patent Application Publication
(10) Pub. No.: US 2007/0065473 A1
(43) Pub. Date: Mar. 22, 2007

(54) Nitric oxide gas (gO) as a cosmetic and wound healing agent

(76) Inventor: Christopher C. Miller, North Vancouver (CA)

Correspondence Address: SIDLEY AUSTIN BROWN & WOOD LLP (LAIP GROUP), 555 W. FIFTH ST., SUITE 4000, LOS ANGELES, CA, 90013, US

(21) Appl. No.: 11/497,557

(22) Filed: Aug. 1, 2006

Related U.S. Application Data
(63) Continuation-in-part of application No. 10/615,546, filed on Jul. 8, 2003.
(60) Provisional application No. 60/431,876, filed on Dec. 9, 2002.
(60) Provisional application No. 60/394,690, filed on Jul. 9, 2002.
(60) Provisional application No. 60/409,400, filed on Sep. 10, 2002.

(51) Int. Cl. A61K 8/97; A61K 8/19

(52) U.S. Cl. 424/401; 424/47; 424/74

(57) ABSTRACT

The present invention provides a method and device for exposing injured mammalian tissues, in a non-abrasive manner, to an effective amount of exogenous gaseous nitric oxide (gNO) in order to promote healing by reducing the size, duration and severity of wounds as well as controlling the infection by reducing number of pathogens at the site and the surrounding area. The present invention also provides methods of cosmetically treating the skin wherein a cosmetic agent in combination with gNO is applied to the skin.

### CLAIM OF PRIORITY

This application claims priority to U.S. application Ser. No. 10/615,546 (filed Jul. 8, 2003), which claims priority to provisional Patent application Nos. 60/431,876 (filed Dec. 9, 2002), 60/394,690 (filed Jul. 9, 2002), and 60/409,400 (filed Sep. 10, 2002). These applications are incorporated by reference as if set forth herein.

### FIELD OF THE INVENTION

This invention pertains to a method and device for delivery of gaseous nitric oxide (gNO). The gNO is directed to a wound on a mammal to promote the healing of the wound. The gNO is also directed to the face, hands and body as a cosmetic therapy to treat wrinkles, acne, dryness, dullness, and general aesthetic appearance of the skin.

### BACKGROUND OF THE INVENTION

Nitric oxide (NO) is an intensely studied molecule in medical science. It is a short-lived free radical. It is also highly reactive and locally diffusible because of its small molecular size and unpaired electron. Since its discovery as an endothelium derived relaxing factor in 1987, it has become evident that NO is a widely distributed multi-functional intra- and inter-cellular messenger. NO is formed from a terminal nitrogen atom of arginine through an oxidation process with molecular oxygen. It is understood that certain enzymes, referred to as nitric oxide synthases (NOS), are responsible for that oxidation process.

NO has also been shown to have a direct or an indirect role in pathophysiology of numerous bodily functions both in human and mammals. Some of these bodily functions and disorders include but not limited to (1) blood flow and pressure in body circulatory system, (2) pulmonary hypertension, (3) asthma, (4) inflammatory response, (5) infection, (6) cancer, (7) angiogenesis, (8) neurotransmission in nervous system, (9) diabetes, and (10) sexual dysfunction such as penile erection. Over the past several years, NO has also been noted to play an important role in wound healing.

Conventionally wounds heal through a three step process. The first step is called an initial inflammatory phase. This phase is defined by platelet aggregation, degranulation, and phagocytosis.

The second step is referred to as the proliferative phase. This phase is characterized by an expansion of reparative cells. The reparative cells include fibroblasts. Fibroblasts are a major synthetic element in a wound, and are responsible for production and reorganization of structural proteins (such as collagen) required for tissue repair. Endothelial migration and angiogenesis also initiate in this stage.

The third and last step is called the maturation phase. This phase is the longest stage in the wound healing process. In this phase, newly deposited collagen (from fibroblasts) and an extracellular matrix are reorganized and result in increasing wound strength and eventually in mature scar formation.

NO is both directly and indirectly, as a regulator, involved in each of these physiological steps. In fact, many wound resident cells have the ability to synthesize or affect the synthesis of NO. Examples of wound resident cells include and are not limited to macrophages, neutrophiles, endothelial cells, vascular smooth muscle cells, keratinocytes, lymphocytes, and fibroblasts.

Lack of NO and arginine in mammals result in a decrease in (a) NO metabolism, (b) wound breaking strength, (c) collagen synthesis, (d) epithelialisation, and (e) wound contraction. In complementary studies that used chemical NO donors and arginine rich diet, the results point toward an increase in all of the above factors, which result in the promotion and acceleration of the wound healing.

NO is also a known factor in promoting angiogenesis (development and rearrangement of new blood vessels within an injured tissue), increasing circulation to injured site, stimulating collagen synthesis in fibroblast, and mediating growth factor release. There are many situations a wound's healing response is delayed or inhibited in patients with systemic diseases. Systemic diseases include and are not limited to liver failure, renal impairment, diabetes, peripheral vascular disease, or in patients taking drugs like corticosteroids or immunosuppressive agents that inhibit healing, or prolonged process of healing in elderly. In all these cases, additional exogenous NO gas enhance the healing process.

Keloids and hypertrophic scars are examples of scarring pathology that is characterized by excess collagen deposition during process of wound haling. The exact mechanism of this disorder is not well understood, but it is shown that NOS expression and NO production are significantly reduced in fibroblasts derived from hypertrophic scars. By maintaining high levels of NO in these wounds, exogenous gNO can offer a potential treatment.

There are many situations in which the healing response in a wound is delayed or inhibited in patients with systemic diseases. In all these cases, additional exogenous gNO can potentially enhance or accelerate the wound healing process. One of these areas that gNO can have vast therapeutic impact is patients with diabetes dealing with complicated non-healing wounds. As mentioned above, a systemic deficiency of endothelial-derived NO has been observed in diabetics, suggesting that NO plays a fundamental role in the patahogenesis of chronic, non-healing wounds. Diabetes affects an estimated 15 million people in the US alone.

In flap and micro-surgery reperfusion to ischemic tissue and organs is a critical criterion in survival of the tissue. Therefore administration of exogenous gNO, due to its vasodilatory and angiogenesis effects, can potentially maintain the vascular tone and protect the skin flap.

Secondary infection in chronic and open wounds can seriously slow down or complicate the process of healing. NO antimicrobial has been well documented in literature and supported by applicant's in vitro and animal studies using gNO. Nitric oxide has clearly shown bactericidal and/or bacteristatic effects on at least two of the most common pathogens in chronic wounds, namely pseudomonas auroginosa and staphylococcus aureus.

In PCT International Application number PCT/CA99/01123, the assignee of this application disclosed a method and device for treatment of respiratory infections by NO gas inhalation. This property of NO is critical in controlling an infection and giving the immune system a chance to fight and clear out the pathogens. In U.S. Pat. No. 6,432,077, Stenzler discloses “device and method for treatment of surface infections with nitric oxide.”

In view of the above, there is a need in the art for therapies that improve and accelerate the healing process in wounds through a temporally regulated manner, with specific attention to chronic wounds such as non-healing diabetic foot ulcers, 3^{rd}degree burns, venous and pressure ulcers, and hypertrophic scarring and keloids.

### SUMMARY OF THE INVENTION

The present invention provides a method of applying gaseous NO to the face, hands and/or body as a cosmetic therapy to treat wrinkles, acne, dryness, dullness, and general aesthetic appearance of the skin. NO gas may be applied in combination with a cosmetic agent.

The present invention provides a method and device for exposing injured mammalian tissues, in a non-abrasive manner, to an effective amount of exogenous gaseous nitric oxide (gNO) in order to promote healing by supporting skin cell growth, angiogenesis and tissue perfusion, and reducing the size, duration and severity of wounds. gNO may be applied with an agent and/or one or more growth factors.

### BRIEF DESCRIPTION OF THE DRAWINGS

FIG. 1 illustrates a cross sectional diagram of one embodiment of the gNO delivery wound cover device.

FIG. 2 illustrates an alternate embodiment of the wound cover.

FIG. 3 illustrates one embodiment of an inflatable bathing unit for delivery of gNO to wound covers in FIGS. 1 and 2.

FIG. 4 illustrates schematics of gNO chamber designed to carry out in vitro studies for exposing human cultured cells as well as microorganisms to various concentrations of gNO, under optimal growth conditions.

FIG. 5 illustrates morphology of fibroblast cells exposed inside gNO chamber to less than 200 ppm versus control group inside conventional tissue culture incubator.

FIG. 6 illustrates in vitro growth of fibroblast cell following exposed to 20 and 200 ppm in comparison with control.

FIG. 7 illustrates cell attachment capacity of human fibroblasts following exposure to 160 ppm gNO.

FIG. 8 illustrates wound bacterial content following topical application of 200 ppm gNO in a full thickness infected wound model in rabbits.

FIG. 9 shows wound bacterial content following topical application of 400 ppm gNO in a full thickness infected wound model in rabbits.

FIG. 10 shows rabbit blood serum NOx(NO₂& NO₃) levels following topical application of 400 ppm gNO.

FIG. 11 illustrates rabbit blood methemoglobin levels following topical application of 400 ppm gNO on a full thickness infected wound model.

FIG. 12 shows mRNA expression for collagen and collagenase following exposure to 200 ppm gNO for 24 and 48 hours.

FIG. 13 illustrates histology analysis of full thickness infected wound exposed to 200 ppm gNO for 24 hours.

### DETAILED DESCRIPTION OF THE PREFERRED EMBODIMENTS

It has been found that exogenous nitric oxide gas acts as an initiator of wound healing in mammals. First aspect of the invention encompasses promoting and accelerating the process of wound healing by a topical application of medical grade exogenous nitric oxide gas, in a concentration dependent manner, to an injured tissue (e.g. skin, bone, tendon, ligament, cornea, or other tissues) in a mammal.

In another aspect, the invention provides a method for increasing local blood flow at the wound site or in the immediate vicinity thereof, through an increase in local concentration of nitric oxide. Exogenous nitric oxide gas is a potent and effective vasodilator that can accelerate tissue perfusion and maintain vascular tone at the site of injury. Through this action, it will bring more nutrient, oxygen, inflammatory and healing factors to the injured tissue resulting in faster healing and closure.

In another aspect, the invention encompasses a device for localized delivery of an effective amount of exogenous gNO to the wound site by utilizing a specialized wound cover that covers the surface of the wound, and isolating it from the external environment.

In yet another aspect, it is also the aim of this invention to prevent further infection (secondary infection) by isolating the wound area from external environment using a transparent wound cover device for the delivery of the gas. This also prevents further wound dehydration.

In yet another aspect, gNO therapy is administered topically at the site of the wound immediately post trauma, or applied during a surgery procedure. In case of chronic or non-healing wounds, therapy illustrated in this invention can be administered continuously for as long as 4 weeks.

Briefly stated, the present invention provides a non-abrasive method that will accelerate and improve wound healing, particularly in situations where complicating factors are present such as, and not limited to, diabetic conditions, foot ulcers, venous and pressure ulcers, post surgery hospital acquired infectious wounds, non healing wounds in elderly and/or immunocompromised, keloids, hypertrophic scarring, burns, and skin flaps. In particular, it is believed that the present invention promotes wound strength and healing by activating the production of fibroblasts, stimulating synthesis of collagen, and initiating angiogenesis. It is also the aim of this invention to increase the local blood flow to the site of injury and by doing so, bring more nutrients and oxygen to the wound.

The present invention describes a new method and device for improving and accelerating the healing process of wounds in mammals, with particular attention to nonhealing and chronic wounds such as diabetic conditions, foot ulcer, venous and pressure ulcers, wounds in elderly and immunocompromised, and 3rd degree burns. A preferred embodiment of the present invention delivers an external source of nitric oxide gas at an effective dose range and for optimal duration to the injured tissue such as, but not limited to, skin, bone, tendon, ligament, and cornea.

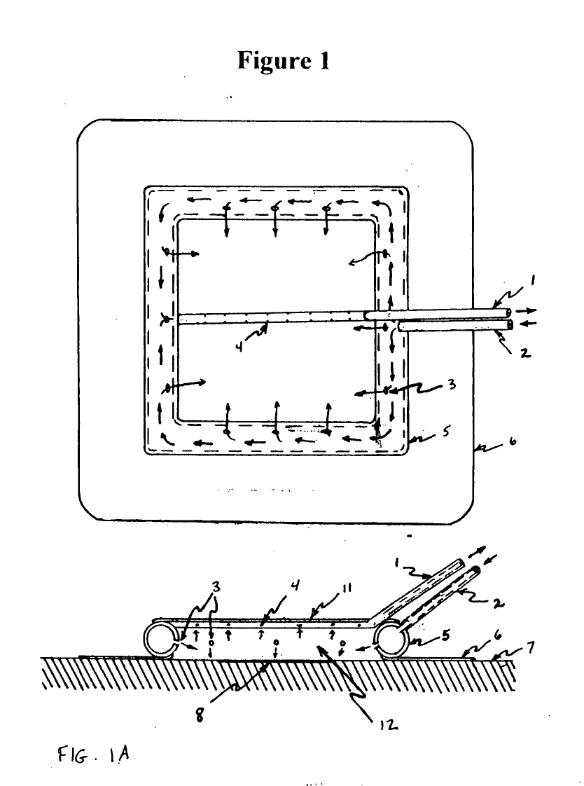

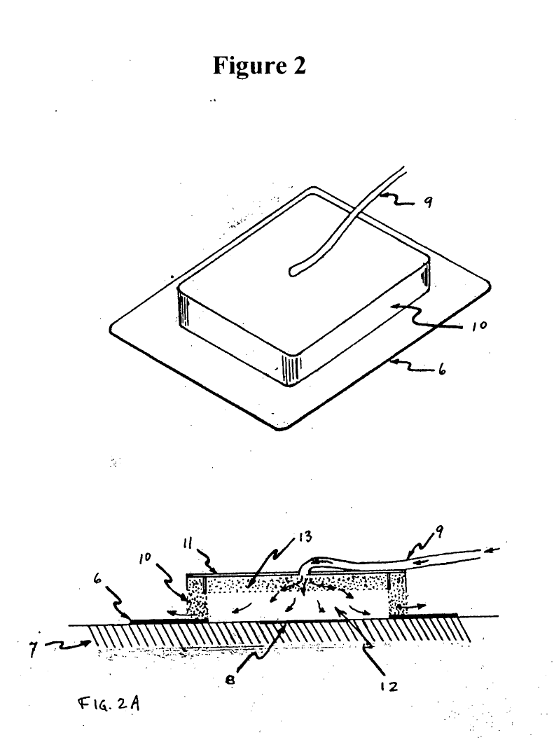

Methods and apparatus for delivery of exogenous nitric oxide gas from an external source to the wound cover (FIGS. 1 and 2) are disclosed herein and other methods have been disclosed in U.S. Pat. No. 6,432,077, and commonly assigned PCT International Application number PCT/CA99/01123. Applying gNO promotes wound healing in bodily injuries or lesions, in mammals. The injuries or lesions can be caused by physical means such as mechanical, chemical, viral, bacterial, or thermal means, which disrupt the normal continuity of structures.

The proposed therapy of this application facilitates the process of wound healing through suppression of inflammation, and the stimulation of cellular viability and proliferation which will lead into an increase in wound breaking strength, collagen synthesis, epithelialisation, and wound contraction.

In order to be effective in enhancing wound healing, the local concentration of nitric oxide in the injured tissue is increased through continuous or intermittent exposure to an effective dose of gNO. Dose level and duration of exposure will vary depending on nature and extent of the injury and will be assigned by those skilled in the art. Therapeutic dose of gNO will vary from 20 parts per million (ppm) to 1,000 ppm. The time for treatment will vary from 1 to 8 hours intermittent exposures daily or continuous exposure ranging from 1 to 31 days.

gNO therapy is administered topically at the different site of the wound immediately post trauma, or even applied during a surgical procedure. In case of chronic wounds, e.g., diabetic conditions, foot ulcer, tennis elbow, jumper's knee, or in non-healing wounds, this therapy will be prolonged by those skilled in the art until desired healing effect has been obtained. It is hereby suggested that prolonged exposure, in excess of 8 hours, and from 1 to 4 weeks, to an effective dose of exogenous gNO has a therapeutic effect in treating conditions dealings with complicated or non-healing wounds.

A gNO wound cover is preferred over other types of wound covers (e.g. bandages or dressing) because the same gNO wound cover can be repeatedly connected to a gNO source 1 at a desired exposure interval and temperature and does not need to be changed or removed following gNO therapy. In contrast, repeated application and removal of bandages and/or dressings will increase the probability of microbial infection. Additionally, airtight bandages and/or dressings prohibit moisture from escaping. Thereby bacteria, present at the time of procedure, flourishes under the bandage.

FIG. 1 illustrates a cross sectional diagram of one embodiment of the non-contact gNO wound cover device. The wound cover consists of a sealing lip 6, which attaches to the uninjured skin 7, surrounding the wound 8 completely. The rigid ring 5 forms the wall and a barrier layer 11 attached to the top of ring 5 forms the remainder of the cover thus providing an enclosed treatment volume 12 over the wound. The wound cover is preferably constructed of a clear plastic such as, but not limited to, polyvinyl chloride which is stiff but which may conform to the shape of the surface of the body of the patient. Gases such as gNO, air or other gaseous mixtures are introduced into the treatment volume area 12 by a supply line 2 which flows into a ring 5. Orifices 3 on the inside wall of the ring 5 allow the gas to enter the treatment volume area 12. The spent gas is exhausted through the orifices 4 of an exhaust tube 2. A sealing lip 6 attaches to the skin 7 by an adhesive.

FIG. 2 illustrates an alternate embodiment of the device shown in FIG. 1. In this embodiment of the device, the sealing lip 6 attaches to the uninjured skin 7 by means of an adhesive, surrounding the wound 8 completely. The sides of the ring 10 are in this embodiment constructed of gas permeable but stiff foam which is flexible enough to conform to the patient's body. A barrier layer 11 covers the top of the device. Below and attached to the barrier layer 11 is a layer of gas and liquid permeable porous foam 13 through which the gases introduced through delivery line 9 must pass. The purpose of the porous foam layer 13 diffuses the gas flow. Also through this porous foam layer 13 additional medications or wound healing agents (gas or liquid) may be administered through deliver line 9 in order to alter the environment of treatment volume area 12. The treatment gases introduced into the treatment volume area 12 exhaust through the permeable walls 10 of the device.

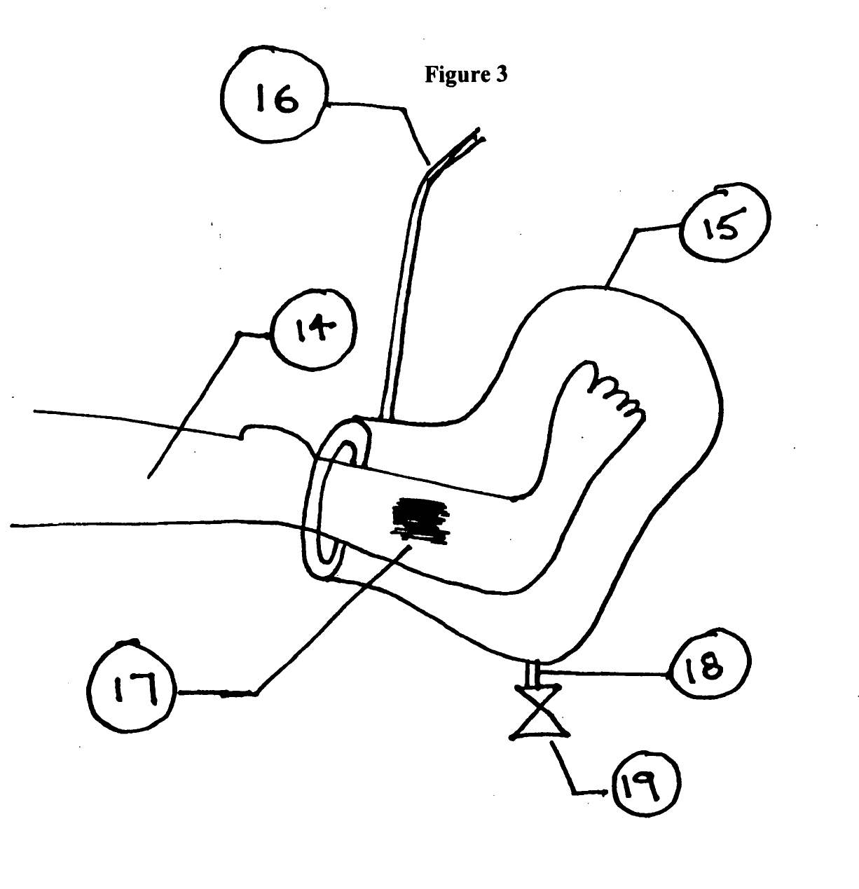

FIG. 3 shows an inflatable bathing unit 15 that can be used on its own or in combination with wound cover shown in FIGS. 1 and 2 when a higher therapeutic dose of gNO is being administrated and the risk of prolonged exposure to nitrogen dioxide (NO₂) should be avoided. The bathing unit 15 can take the shape of a boot that is placed over the patient's foot 14. The inlet line 16 can be connected to a gNO delivery device (e.g. AeroNOx, Pulmonox Medical Inc) titrating the exact amount of gNO desired in therapy. The delivery line 18 in the bathing unit can be connected to delivery line 9 of the wound cover and excess gas mixture can exit the unit through the one way exhaust valve 18. In order to scavenge excess gNO and nitrogen dioxide (NO₂), a specialized filter 19 is attached to the exhaust valve 18 (e.g. No. 67-35-813, Drager Industrial Ltd).

It is understood by those skilled in the art that although the embodiments of the devices illustrated in FIG. 1 and FIG. 2 are rectangular in shape, the device could be supplied in a variety of shapes and sizes. Additionally the size of the required treatment volume and the size of the wound will determine the size of the sealing lip 6 and the size and thickness of the ring 5 and barrier layer 11 to prevent contact with the wound. It would also be understood by those skilled in the art that other embodiments of the device can also be produced to include alternative methods of dispensing of the spent gases such as through filters in the barrier layer, or construction of the device as a number of parts that could be assembled as required to form any desired shape or size without departing from the scope of the invention.

Devices in FIGS. 1 and 2 when used with gases such as gNO and air mixtures, complete exchange of the treatment volume at a rate of three times per minute produced negligible formation of NO₂ with treatment volumes of 200 milliliters. The size of the device and treatment volume will determine the flow of gas necessary to maintain minimal formation of NO₂.

In another aspect of the invention, gNO can be delivered locally as a spray stored in small pressurized cylinders at a preset concentration immediately following a trauma, where gNO delivery system and wound cover are not accessible.

Due to the active role of nitric oxide in various physiological processes, for optimal use of the present invention, gNO should be delivered locally, i.e., take within or in the immediate vicinity of an injured tissue. Nitric oxide is highly reactive with air oxygen and iron molecule in heme moiety of hemoglobin leading to production of NO₂ and methemoglobin, respectively. Levels of these two by-products will be monitored closely in sampled air from the wound cover by a chemiluminescence analyzer (e.g. AeroNOx, PulmoNOx Medical Inc.) and blood for the duration of gNO therapy.

Particular pretreatment methods can be particularly advantageous prior to or in conjunction with gNO therapy. For example, gNO therapy can be preceded by mechanically scraping the surface of the wound in order to remove necrotic tissue and debris from the wound surface and increase the penetration power of gNO molecule into the injured area. The invention may also be used in combination with various agents including antibiotics, anesthetics, analgesics, anti-inflammatory agents such as corticosteroids and nonsteroidal anti-inflammatory agents, antiviral agents, vasodilators or vasoconstrictors, antihistamines, other hormones such as estrogens, progesterone, androgens, antiseborretic agents, other cardiovascular agents, mast cell stabilizers, scabicides or pediculicides, keratolytics, lubricants, narcotics, shampoos, burn preparations, cleaning agents, photosensitizing agents, wet dressings and other wound care products in order to further enhance the healing process. Other agents may be employed in combination with gNO therapy to indirectly enhance the local amount of nitric oxide, e.g., by enhancing absorption or prolonging therapeutic effects (such as phosphodiesterase inhibitors), and/or to enhance the activity of NO synthase, or to protect NO from degradation.

Gaseous NO may also be applied to the skin or tissue to treat a wound in combination with one or more growth factors. The growth factors may be supplied to the injured tissue before, after or simultaneously with the gNO. Naturally produced growth factors are instrumental in wound healing, See e.g., Frank, et al., “Nitric oxide drives skin repair: Novel functions of an established mediator,” Kidney International, Vol. 61 (2002), pp. 882-888. Protein-type growth factors are well established to orchestrate cell movements at the wound site. Such growth factors include: FGF, fibroblast growth factor; KGF, kerantinocyte growth factor; PDGF, platelet-derived growth factor; IGF, transforming growth factor; VEGF, vascular endothelial growth factor; EFG, epidermal growth factor; CTGF, connective tissue growth factor; GMCSF, granulocyte-monocyte colony stimulating factor. All of the above growth factors are suitable alone or in combination with gNO.

Another method of the invention is a method of cosmetically treating the skin, comprising applying gNO to the skin and a cosmetic agent. Cosmetically treating refers to any aesthetic treatment of the skin, such as anti-aging, anti-wrinkle, anti-roughness, growth promotion, anti-acne, moisturizing, and cleansing. The target area may be the face, hands, feet, and any body surface. The methods may include applying the gNO before, after or simultaneously to the skin with the cosmetic agent. Moreover, the application of gNO may be combined with treating the skin with steam such that skin pores may be opened and made more accessible to gNO and/or the cosmetic agent. Application of steam to the skin may be made before or simultaneously with treatment with gNO and/or cosmetic agent. While moderately heated steam is beneficial, the steam should not be too hot that it will burn the skin.

The cosmetic agent of the present invention may be cosmetics, drugs, quasi-drugs, medicines for external use, etc. No particular limitation is imposed on the form of the cosmetic agent, and it may be used in various forms, for example, toilet waters, emulsified compositions, moisturizing creams, cleansing creams, massaging creams, face cleansing creams, packs, beauty lotions, etc.

Included with the cosmetic agent may be publicly known cosmetic components and drug components, for example, water, alcohols, surfactants, preservatives, perfume bases, coloring matter, various kinds of medicinally-effective components, etc. so far as no detrimental influence is thereby imposed on the effects of the present invention.

Included as a cosmetic agent in particular are those active ingredients which effect cleaning and care of the skin and maintenance of a healthy skin condition, external protection of the skin from damaging environmental influences, climatic and actinitic influences, thus in particular with excessive solar and UV radiation, protection of skin from laundry and cleansing agents as well as other environmental stress, thus in particular dust and emissions. Belonging to these in particular are unsaturated fats having a pliable effect, liquid fatty acid esters and hydrocarbons with short-chain branching which have a spreading effect, covering and protective fats, in particular comprising oils, liquid fatty alcohols, silicon oils, solid fatty acid esters and/or fatty alcohols, whereby preferably an oil and/or an oil constituent, in particular also the unsaponifiable portions of a plant oil, such as preferably avocado oil, olive oil and/or at least one native oil is contained as a cosmetic active ingredient in the cosmetic composition according to the invention. In addition, vitamins, oligoproteins, collagen-hydrolysates and known and useful UV filter substances are also to be mentioned as cosmetic agents.

Preferred examples of such a cosmetic agent increasing the skin moisture are generally N-acyl-alkanolamines, preferably lactamide MEA, oleamide MEA and/or acetamide MEA, and in particular N-acyl-ethanolamines, so in particular N-acetyl-phosphatidylethanolamine, N-acetyl-ethanolamine already mentioned several times hereinabove, N-oleoyl-ethanolamine, N-linolenoyl-ethanolamine as well as N-acyl-ethanolamine and/or N-acyl-2-hydroxy-propylamine, whereby the latter two ingredients contain fatty acids of cocoa fat and/or palm oil as acyl residues. Also effective as a cosmetic agent are the fatty acid salts of betaine.

Further, depending on the type of cosmetic agent, the cosmetic agent may be used with at least one preservative, antioxidant, thickener, gel-forming agent and/or alcohol, preferably a multivalent alcohol.

The cosmetic agent may be one of the following categories of agents, such as antioxidants. Also listed herein are types of agents that are typically present in combination with the active cosmetic agent, such as a binding agent.

Antioxidants: Our environment can severely damage our skin by oxidizing and deteriorating our cells, like rust on a car, in the form of free radicals. Antioxidants fight free radicals, and prevent much of this damage.

Binding Agent: Substances that hold products together and prevents separation of the water and lipid components.

Emollients: Substances that smooth and soften the skin. There are literally several hundred emollients, each providing its own individual texture to the skin.

Emulsion: A blend of oil ad water into a single smooth product.

Humectants: Substances that can attract water, usually out of the air. By definition, all humectants are also moisturizers.

Lubricants Substances that make skin feel smoother to the touch and reduce friction.

Preservatives: Substances that kill detrimental bacteria, yeast and/or molds, thus prevent spoilage.

Solvents: Substances, such as alcohol or water, which dissolve other ingredients.

Surfactants Substances that enable a topical product to easily spread and glide across the skin.

Vehicle: The base that carries the active ingredients.

Common cosmetic agents also include the following, which may also be termed the active ingredient.

Acetate: an acetic acid salt; the word that follows or precedes acetate on an ingredient list determines the function.

Acetone: solvent commonly used in fingernail polish removers and toners; can be drying and irritating depending on concentration. Non-acne forming.

Acetylated Lanolin Alcohol: helps soften skin and has anti-allergenic tendencies; is highly prone to comedogenicity (causes blackheads and/or whiteheads) this is an example of an alcohol that is not drying.

Acrylates Copolymer: active ingredient in an oil-absorbing gel.

Acrylates/Octylpropenamide Copolymer: creates a water-repelling basis for cosmetics claiming water-proof properties.

Alcohol SD-40: sometimes listed as SD Alcohol 40 and synonymous with alcohol SDA-40, it is a high grade purified cosmetic alcohol. Evaporates instantly, so it is used as a vehicle to transport important ingredients to the skin's surfact and then leave then there; gentler to the skin that ethyl (rubbing) alcohol. May help kill bacteria.

Algae/Seaweed Extract:B an emollient, restoring moisture content to skin; claims to have antioxidant properties.

Allantoin: a botanical thought too have calming properties to the skin that help resolve irritation.

Alpha Hydroxy Acid: an active ingredient derived from fruit acids. Helps exfoliate the top layers of the epidermis; promotes moisture restoration and helps penetration of other ingredients; highly sought after for use in anti-aging and bleaching skin care products. May irritate the skin but do not advance skin aging. Buffering the pH helps make them less irritating to the skin. AHA's include: citric acid (citrus fruits), glycolic acid (sugar cane), lactic acid (milk), and the less common AHAs used in cosmetic malic acid (apples) and tartaric acid (wine). AHAs increase sun sensitivity due to their exfoliant behavior.

Alpha Lipoic Acid: an antioxidant; is both water and fat soluble so it can go to many areas of a cell.

Alum: usually in crystal or powder form; has strong astringent properties; used in stypic sticks, popular with men who often nick themselves shaving.

Ascorbic Acid: vitamin C; used as an antioxidant in its L-ascorbic acid form, can have skin lightening effect in certain preparations, is important to keeping as well as rebuilding healthy collagen fibers.

Ascorbyl Palmitate: preservative with antioxidant properties: its presence helps make possible the blending of vitamins A, C, and D into a suspension.

Beeswax: waxy component in many skin care products; provides moisture as well as a barrier mechanism. Allows for oil and water blending (emulsion).

Benzoyl Peroxide: antibacterial agent kills p. acnes, the germ responsible for acne flares. Can be drying and/or irritating. Some reports of benzoyl peroxide skin allergies. Available in both prescription and OTC forms ranging from 2½-10%.

Beta Hydroxy Acid: new term for salicylic acid, a long term ingredient used for exfoliation of dry skin as well as for acne therapy.

Boric Acid: used as a preservative to prevent yeast overgrowth. While an antiseptic agent, it is out of favor for use in skin care products due to its irritancy potential.

Caffeine: used to alleviate puffiness under eyes.

Camphor: a cooling agent used to help alleviate itching and irritation in many skin care and medicated products.

Carbomers (934, 940, 941, 980, 981): stabilize and thicken products.

Carmine: red pigment; found in many mascaras, and it can be irritating.

Caviar (Roe Extract): Fish eggs high in mineral and vitamin content (B1, 2, 6, as well as A, E & D). Promoted as useful for improving the health and appearance of maturing skin.

Cellulose: plant matter used to as a thickener, also helps suspend oil and water formulations.

Ceramides: epidermal hydrating agent; expensive due to its scarcity.

Ceteareth: cetearyl and stearyl alcohols combined for use as a lubricant.

Cetyl Alcohol: lubricant and helps emulsify oil and water formulations. Is non-irritating nor drying and is not related to isopropyl alcohol. Not supposed to be acne-forming.

Collagen: the main supporting fiber located within the dermis, gives strength and provides structure. You cannot replace lost collagen by applying it to your skin as it is incapable of penetration, but topical collagen is able to moisturize and hydrate by holding many times its own weight in water.

Cyclic Acid: a new term for Hyaluronic Acid: a strong hydrating complex holds 1000 times the water in skin.

Cyclomethicone: form of silicone; gives products a smooth texture without blocking pores.

Dimethicone: also a form of silicone; gives slip and glide to products: has been used in some scar therapies.

EDTA: preservative: potential for causing contact dermatitis.

Elastin: a fiber within the dermis similar to collagen, gives support and “snap” to the skin. In topicals, it cannot penetrate the skin, but is great for protecting against moisture loss.

Ellagic Acid: this naturally occurring ingredient helps to inhibit the formation of sun and age spots.

Ethyl Alcohol: aka rubbing alcohol or ethanol; antibacterial function; usually too strong for regular use in cosmetics.

Glycerin: hydrates and provides a skin barrier; allows topical agents to go on very smoothly; a concern is clogging of pores when present in high concentrations.

Glycine: amino acid vital to collagen composition and production.

Glycogen: building block of sugar, acts as a conditioner.

Glycolic Acid: an alpha hydroxy acid helpful for acne-prone skin, resolves dry skin conditions; used in chemical peels as well as to help reduce the appearance of pores and wrinkles; exfoliates excess flaking or crusty skin.

Glycol Stearate: thickening agent helps give products a luminescent or opalescent appearance.

Grape Seed Extract: a botanical extract shown to be an effective antioxidant.

Green Tea Extract: shown to be a powerful antioxidant and is found in Replenix.

Hyaluronic Acid: lately referred to as a “cyclic acid”; can hold 1000 times its own weight in water, great hydrator as used in Cellex-C Hydra 5 B-Complex and Skin Ceuticals Hydrating B5 Gel; also helps to draw in “active” ingredients deeper into the skin.

Hydroquinone: skin pigment lightening agent; a maximum of 2% may be obtained over the counter; higher concentrations available by prescription.

Ispropyl Alcohol: vehicle with antibacterial properties; drying to the skin especially in higher concentrations.

Isopropyl Isostearate: emollient.

Isopropyl Palmitate: emollient helps moisturize skin. No allergic potential although is derived from palm and/or coconut oils. Comedogenic in nature.

Isostearic Acid: fatty acid that forms film on skin.

Kaolin (China Clay): used in oil-absorbing powders and masques; highly absorbent.

Kojic Acid: skin lightener; touted as a bleaching agent for ethnic skin.

Lactic Acid: alpha hydroxy acid used in dermatology to hydrate and smooth dry, flaking skin. May occasionally be used in higher concentrations (well above 12% medical grade) as a chemical peel.

Lanolin: emollient and moisturizer; obtained from sheep; a sensitizer like other wool derivatives, in eczema-prone individuals.

Lecithin: a water-attracting agent used in products to help hydrate the skin and improve the texture and ease of spread onto the skin.

L-Ergothioneine: naturally occurring antioxidant.

Licorice Extract: skin lightener; deemed as more potent than kojic acid or vitamin C for this function.

Linoleic Acid: used to create emulsions; its EFA origins help to hydrate dry, parched skin. Cosmetic vernacular refers to it as Vitamin F.

Lysine: amino acid incorporated to condition skin.

Octyl Methoxycinnamate: FDA approved chemical sunscreen with contact dermatitis potential in some individuals. Related to Balsum of Peru with cross reactions possible for those with contact dermatitis to either agent.

Octyl Palmitate: allows hydration and works as a solvent without giving skin a greasy texture.

Octyl Salicylate: Commonly incorporated into sunscreens for its antibacterial abilities and helps prevent product from turning rancid.

Oxybenzone: FDA approved UVA absorbing chemical sunscreen ingredient.

PABA (Para-Aminobenzoic Acid): UVB absorber used in sunscreens during the 1970's; became a frequent cause of contact dermatitis, therefore it is now out of favor.

Panthenol: a B vitamin (B5), works as a humectant (holds water in the skin). May promote healing.

Parabens: preservatives; deemed safe and unlikely to irritate the skin. widely used for cosmetics; various forms will be listed with the ingredient usually ending in “-paraben”, as in the following word (i.e. methyl paraben).

Petrolatum: heavy bland base, most commonly known for its use in Vaseline; good for sensitive skin however it is occlusive and can cause plugging of the pores and acne in prone individuals.

Polybutene: helps make liquids texturally viscous.

Poly Hydroxy Acid: PHA, derived from the buds of fruit trees, claims to be gentler yet as effective as AHAs; still debatable.

Proline: amino acid vital to the composition and production collagen.

Propylene Glycol: vehicle for cosmetic solutions; excellent for hydrating dry skin but can act as a contact dermatitis sensitizer in prone individuals.

Resveratrol: antioxidant which supports and protects collagen.

Retinol: a derivative of vitamin A; fat soluble; depending upon concentration, estimated to be approximately 10 times less effective than tretinoin.

Retinyl Palmitate: (also known as Vitamin A Palmitate); considered a more stable alternative to retinal for normalizing the skin's texture and helping smooth out fine lines. Is the ester of retinol combined with palmitic acid; thought to be less irritating than retinol.

Retinyl Palmitrate Polypeptide: water soluble formulation of Vitamin A.

Rose Hips: botanical extract of rose petals found to have high concentrations of vitamin C.

Salicylic Acid: classified as a BHA (beta hydroxy acid); medically used as an exfolliant and debriding agent. Cosmetically used in some chemical peels and to reduce oiliness, acne and the appearance of fine lines.

Silica: highly oil absorbant.

Silicone: protects the skin and creates a sheen. Thought to be helpful in reducing the appearance of hypertrophic scars.

Silk Powder: incorporated into cosmetic powders to help absorb skin moisture and oils.

Silk Proteins: prevents dehydration; commonly found in eye rejuvenation creams.

Sodium Bicarbonate: neutralizes acid, making products less irritating; commonly known as baking soda.

Sodium Borate: preservative; related to boric acid; potential irritant.

Sodium Hyaluronate: related to Hyaluronic acid (salt form), works to moisturize the skin; can hold more than 1000 times its own weight in water.

Sodium Laurel Sulfate: used in most cleansers and soaps; acts as a surfactant, offers good foaming qualities; a known skin irritant, but contrary to popular urban myths, does not cause cancer.

Sorbic Acid: preservative; primarily protects product from yeast overgrowth.

Sorbitol: sugar-based ingredient; pulls water by osmosis from the largest source. Typically this is the air, so it helps hydrate skin. In arid conditions, however, water will be pulled out of the skin, resulting in dehydration.

Stearic Acid: Essential fatty acid used in soap manufacturing; may cause irritation.

Sulfur helps kill normal bacteria on the skin improving acne, seborrhea and psoriasis conditions. Typically found in soaps, shampoos and some topical acne medications.

Titanium Dioxide: physical UV blocker, helps block both UVA and UVB wavelengths of light.

Triclosan: used as a preservative; felt to be hypoallergenic.

Tyrosine: amino acid that stimulates fibroblasts to make more collagen when paired with ascorbic acid; plays a role in melanin formation.

Vitamin A: useful for smoothing out dry skin, minimizing pore size as well as keeping pores clean, and helps reduce the appearance of fine wrinkle lines.

Vitamin B: helps improve chemical effectiveness; found in products such as Cellex-C Hydra 5-B.

Vitamin C: stimulates fibroblast activity to produce collagen; vital antioxidant both systemically as well as when used topically in L-ascorbic acid form. Typically considered water soluable.

Vitamin D: regulates cell turn over; used in prescription derivatives to help control psoriasis (Dovonex).

Vitamin E: Tocopherol; antioxidant; helps prevent ultraviolet light damage to the skin, so is incorporated into a variety of cosmetic as well as sunscreen preparations. Helps to moisturizer skin as it is oil-soluable.

Water: Most frequently listed main ingredient in skin care products, used in its purest form, void of minerals and other chemicals, hence the various names like distilled, deionized, purified, etc.

Witch Hazel: botanical with astringent properties, helps remove excess surface skin oils.

Xamthan Gum: thickening agent.

The types of tissue that may be treated using methods in the present invention include without limitation human and other mammalian muscle, tendon, ligament, skin, mucosa, bone, cartilage, and cornea. The tissue may be damaged by surgical incisions, trauma (mechanical, chemical, viral, bacterial, or thermal in nature), or other endogenous pathological processes. Healing may be impaired as a result of systemic diseases.

Foot ulcers are a potentially serious complication in diabetics as the healing process is inhibited by a decrease in wound capillaries, fibroblasts, and collagen at the wound site and by immune system's inability to fight infection. The present invention elevates the synthesis of collagen through production of wound fibroblast at the injured site. Through vasodilatory action of gNO, the present invention increases the local blood flow and perfusion to the extremities where the wound is located.

### Experiments

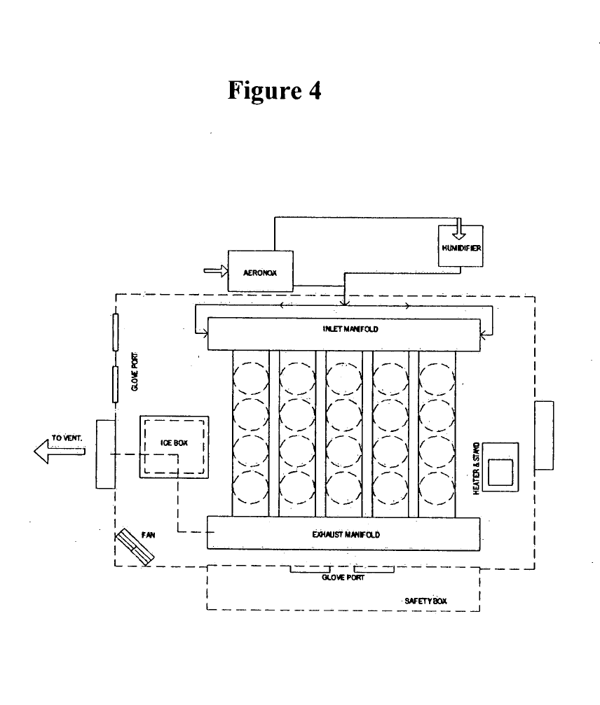

Device: FIG. 4 shows a specialized gNO incubation chamber designed for conducting in vitro studies on mammalian cell cultures as well as bacterial cells under optimal growth conditions to study the effect of gNO exposure on mentioned cells. gNO chamber allowed control and adjustment of following factors in all in vitro studies: gNO dose, total air flow, NO₂ levels, O₂ levels, CO₂ levels, temperature, and humidity.

Bacterial Study: Suspensions of Staphylococcus aureus and Pseudomonas aeroginosa cells were prepared in Tryptic Soy Broth medium, and then plated onto clear Tryptic Soy Agar (TSA) plates at various dilutions to achieve a countable range. A set of 4 cultured plates was incubated in each treatment and control exposure tubes inside the gNO chamber (FIG. 4) at 37° C. and relative humidity (RH) of 80% for period of 24 hours. Plates from the treated group were exposed to various concentrations of gNO (50, 80, 120, 160, and 200 ppm) mixed with medical air at a constant flow of about 5 L/min. Control plates within the chamber were exposed to only medical air at 5 L/min. Four cultured plates were placed inside conventional laboratory incubator at 37° C. with passive aeration for the duration of experiment. This served as a control for bacterial growth within the gNO exposure chamber. Following the incubation period, a count of colony forming units (CFU) was obtained. The difference in CFU between control and exposed plates were used to evaluate the bactericidal effects of nitric oxide. Results were analyzed using an unpaired student to test.

Fibroblast Study: Fibroblast cells obtained from adult patients undergoing elective reconstructive surgery were cultured in Dulbeco's Modified Eagle's Medium (DMEM), supplemented with 10% fetal bovine serum (FBS) and antibiotic-antimycotic preparation and divided into ten 25 cm² vented culture flasks (COSTAR). Four of these flasks (treated group) were exposed to 20 or 200 ppm humidified gNO inside a specialized NO incubation chamber at 37° C. for 24 and 48 hours. The NO exposure chamber was validated prior to the study to eliminate extraneous variables and ensure optimal conditions for fibroblast cell growth. Another four flasks (control group) were placed inside conventional culture incubator and exposed only to ambient humidified air at 37° C. Two flasks were separately harvested and counted as the number of cells at zero time. Following the treatment, fibroblast cells were harvested and evaluated for morphology, cell count, capacity to proliferate and medium pH.

Animal study: In the animal model, full-thickness cutaneous wounds (Set A: 4 rabbits with Eight 8.0 mm punch biopsies & Set B: 4 rabbits with TWO 50×15 mm wounds) were made on each side of dorsal midline and infected with equal volume of Staphylococcus aureus suspension on DAY 0.On DAY 1, treated groups in A and B were respectively exposed to 200 and 400 ppm gNO for total of THREE days. Set A was exposed for TWO 4 hour sessions, interrupted by 1 hour of rest, inside a specialized restraining exposure chamber. A 24-hour continuous delivery model was used for animals in Set B by design of a specialized wound patch. Control groups were only exposed to medical grade air with corresponding flow rate. FOUR random sample punch biopsies (8.0 mm) were collected on post wounding days 3 and analyzed for bacterial content. Another FOUR punch biopsies from both wound and normal skin tissue were collected for fibroblast viability analysis and toxic effects of gNO.

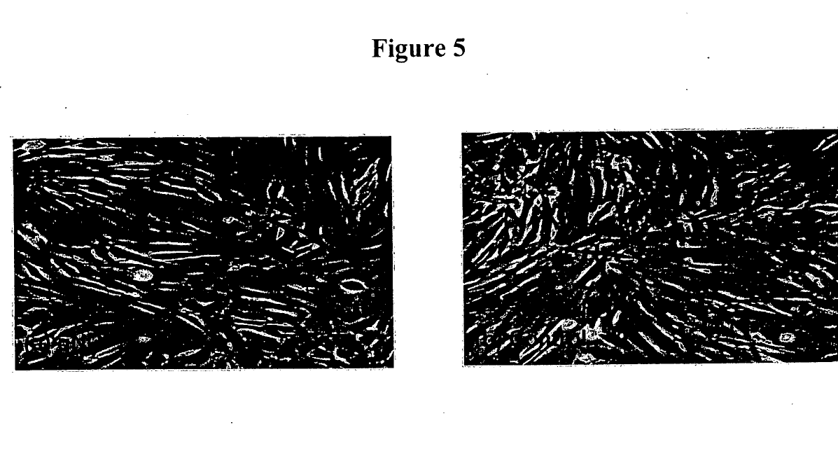

FIG. 5 shows morphology of fibroblast cells from the viability study, where cultured human fibroblast cells were exposed to various gNO concentrations less than 200 ppm continuously for 48 hours. Morphological appearance and attachment capacity of control and treated dermal fibroblasts cells following 48 hours period were quite comparable. Cells under gNO appeared healthy and attached to the culture plates. No toxic effect due to exposure to gNO was observed.

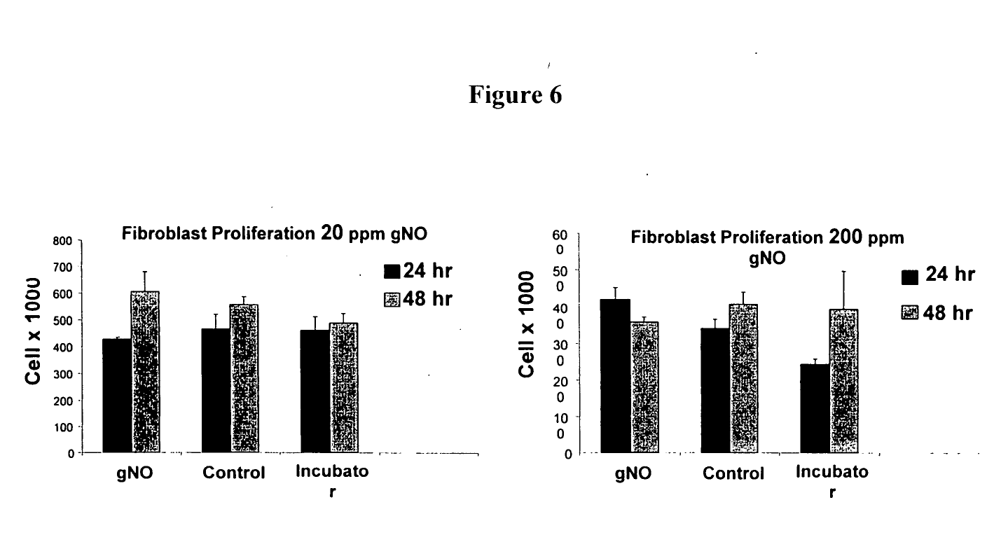

FIG. 6 reveals the results from the cell proliferation assay study. It compared the cellular growth between control and treated group exposed to 20 and 200 ppm gNO for 24 and 48 hours. Again, no significant variation in total cell count of dermal fibroblasts was observed between control and treated groups following 24 and 48 hours exposure to gNO (p<0.05).

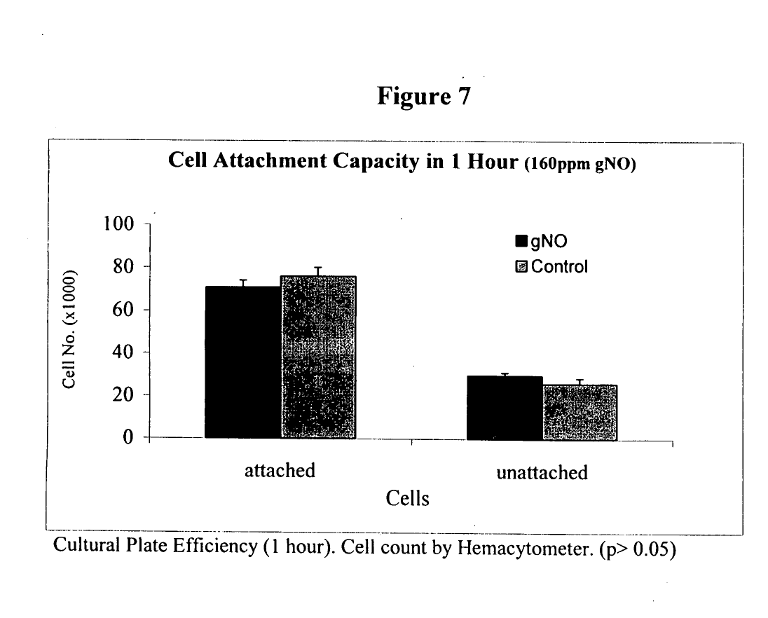

FIG. 7 shows results from Cell Attachment Capacity from the fibroblast cells exposed to 160 ppm of gNO. Capability of cells to reattach to the culture plates within a specified time limit is commonly used as an indication of viability of cells in culture. Both the control and treated groups show a 70% attachment capacity within 1 hour of culturing. This result in conjunction with cell morphology and count support the safety of gNO therapy for topical applications on mammalian skin tissue at least at a range between 100 to 200 ppm of gNO.

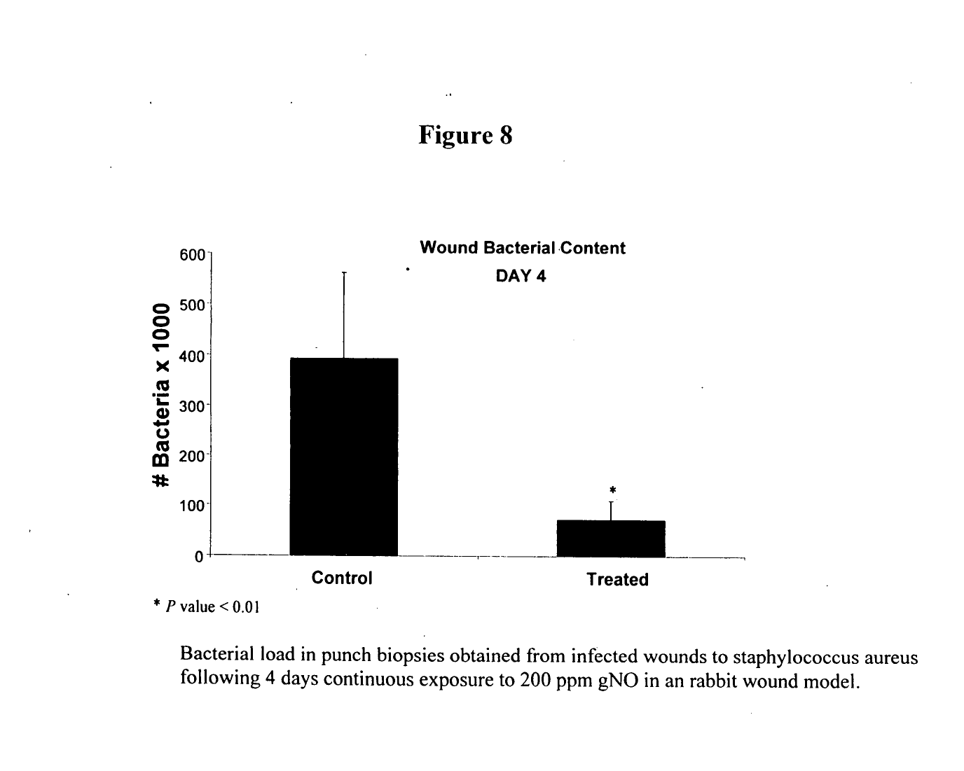

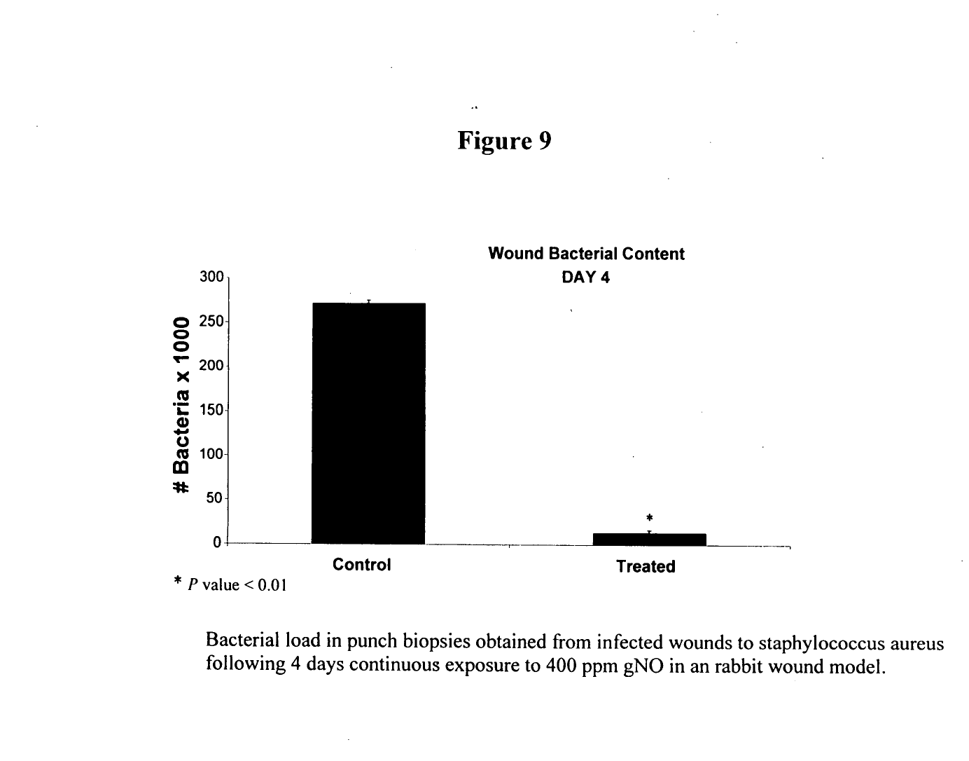

FIG. 8 reveals data from the animal study on bacterial content of the wounds exposed to 200 ppm gNO continuously for 72 hours when compared to control group only exposed to medical air. A significant bacterial reduction is observed in treated wounds. Rabbits appeared comfortable and at ease during the therapy and no toxic effect or damage were observed in the skin of treated animals when compared to the control. A similar device as shown in FIGS. 1 and 2 was used in this study. NO₂ did not exceed safety limits, at any point of the study, set by Occupational Safety and Health Administration (<4.3±0.3 ppm). FIG. 9 shows similar set of data as seen in FIG. 8, but where animal wounds were exposed to 400 ppm of gNO therapy. On average well over 10 fold drop (p<0.05) in bacterial content is observed in comparison between control and treated groups.

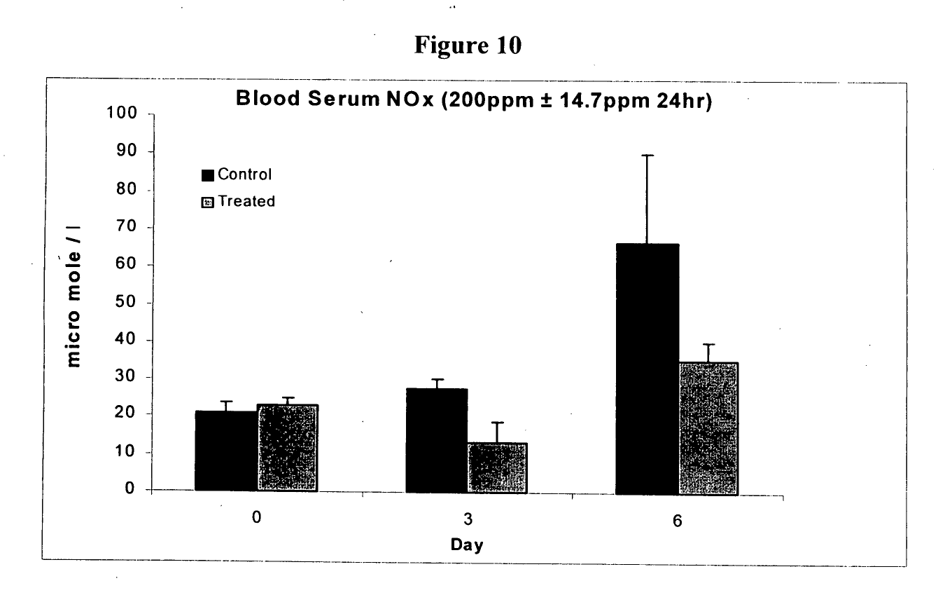

FIG. 10 demonstrates nitrogen oxides levels (NO₂ and NO₃), one of end products of nitric oxide metabolism, measured in blood serum collected from the animals following exposure to 200 ppm gNO intermittently for 6 days. None of the samples show an increased level of NOx due to exposure to gNO indicating the fact that exposing full thickness wounds (8 at 8.0 mm in diameter) will not increase the nitric oxide level in animal's circulation system.

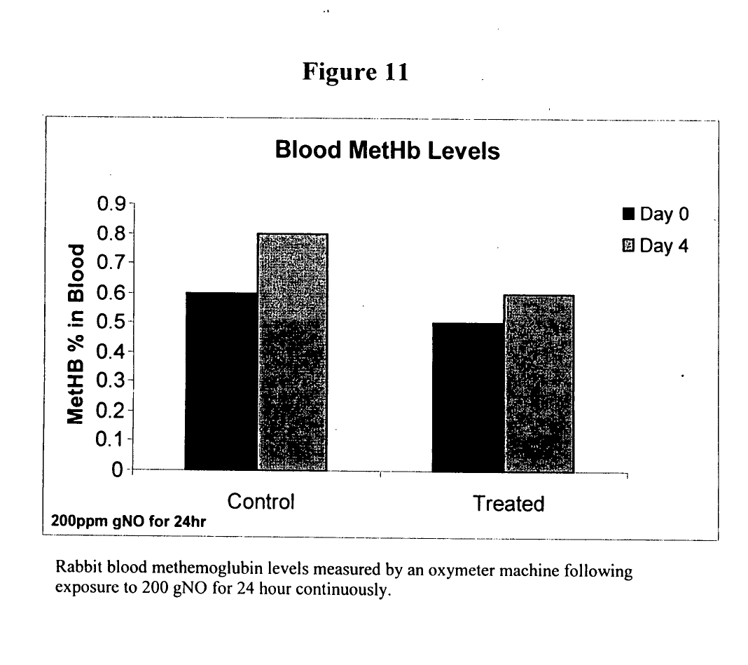

FIG. 11 indicates the level of methemoglobin (MetHb) in animal's blood following 6 day intermittent exposure to 200 ppm gNO. Animals in the treated group did not show an increase level of MetHB in comparison with the control group exposed to air. This further supports the data presented in FIG. 10 to the fact that topical application of gNO on open wounds did not contribute to an increase level of nitric oxide in the circulation.

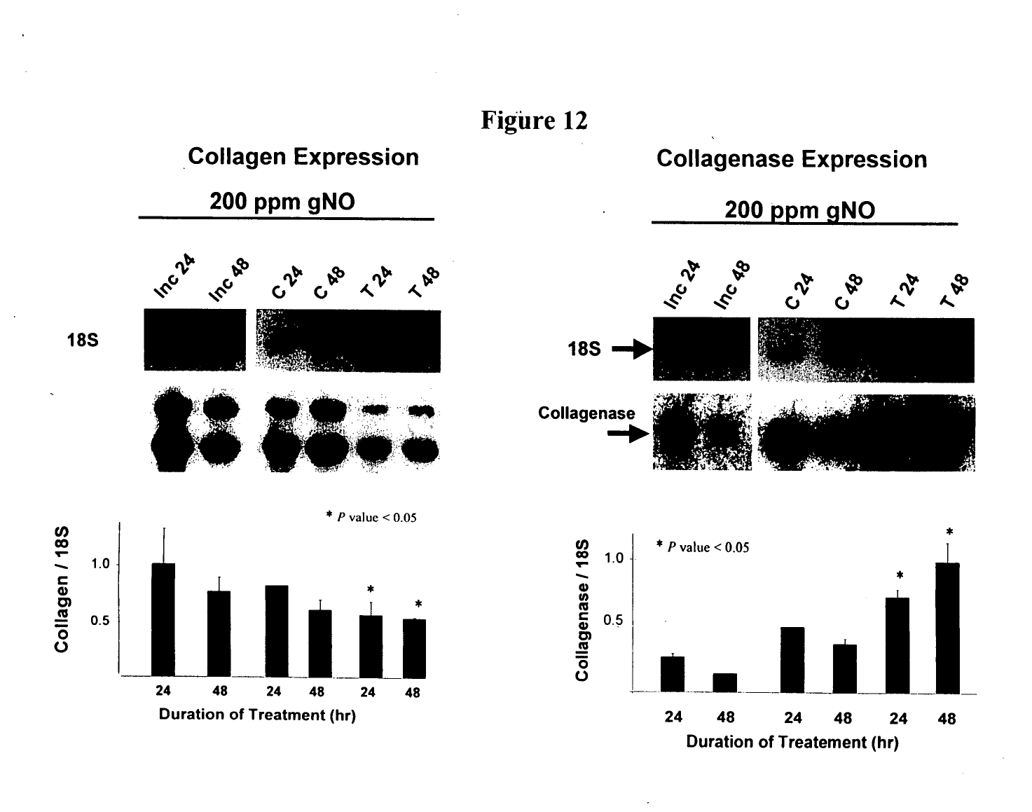

FIG. 12 presents mRNA expression of two important factors in wound healing process, namely collagen and collagenase, treated with high concentration of gNO (200 ppm). A drop in collagen activity is observed at this dose at both 24 and 48 hour exposure indicating a potential for gNO therapy in conditions where excessive healing is present (e.g. hypertrophic scarring). The analysis of collagenase expression further supports the fact that gNO at 200 ppm is not damaging the cellular function, as a significant increase in mRNA activity of this protein is observed.

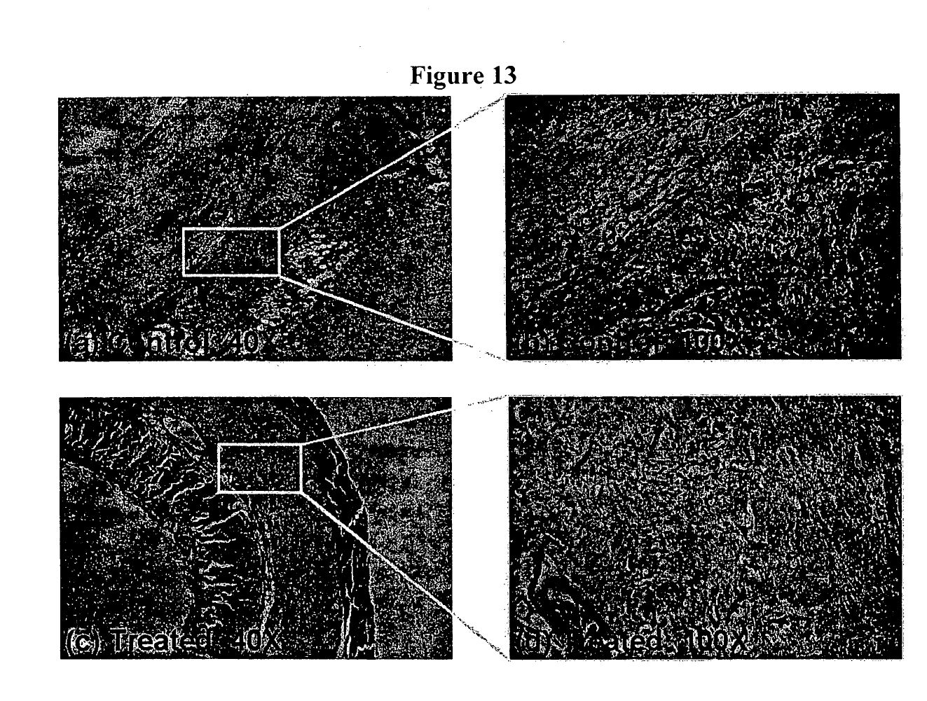

FIG. 13 presents histological analysis of tissue blocks prepared on wound punch biopsies from animals in treated and control groups. Samples from the control group show more advanced neutrophil infiltration and so a higher degree of inflammatory reaction. A lower level of neutrophil concentration is seen in wounds treated with gNO. Wounds treated with gNO show a layer of scab closing on the wound, but control wounds remain open for longer period of time. Overall, a healthier healing process is observed in the wounds treated with gNO. No toxic effects (cellular debris due to apoptosis) can be seen in gNO treated group.

In addition, the role of NO in the survival of tissue (skin) flap has been extremely beneficial. In a free flap, the flap tissue is completely removed from the donor site and attached to the wound by micro vascular techniques. In this case there will be a base that provided circulatory support for the flap.

Nitric oxide synthesized by vascular endothelium is responsible in regulation of vascular tone. Through this action, nitric oxide relaxes vascular tone and increases local blood flow protecting against ischemia-induced flap necrosis.

In flap surgery reperfusion to ischemic tissue and organs is an essential criterion in survival of the tissue. In many surgical procedures this step can lead to intensified tissue injury caused by reperfusion edema. Therefore, administration of exogenous gNO can potentially maintain the vascular tone and protect endothelium cells from the ischemia/reperfusion injury.

Having described preferred embodiments of the invention with reference to the drawings and graphs, it is to be understood that the invention is not limited to these precise embodiments, and that various changes and modifications may be effected therein by one skilled in the art without departing from the scope of the invention as defined in the appended claims.

What is claimed is:

1. A method for cosmetically treating the skin comprising:
applying to the skin gaseous nitric oxide; and
applying to the skin a cosmetic agent.

2. The method of claim 1 wherein the skin is the face.

3. The method of claim 1 wherein the skin is the hands.

4. The method of claim 1, wherein the cosmetic agent is selected from a group consisting of antibiotics, anesthetics, analgesics, anti-inflammatory agents such as corticosteroids and nonsteroidal anti-inflammatory agents, antiviral agents, vasodilators or vasoconstrictors, antihistamines, other hormones such as estrogens, progesterone, androgens, antiseborretic agents, other cardiovascular agents, mast cell stabilizers, scabicides or pediculicides, keratolytics, lubricants, narcotics, shampoos, burn preparations, cleaning agents, photosensitizing agents, and wet dressings.

5. The method of claim 1, wherein the cosmetic agent is selected from the group consisting of vitamins, betaine, antioxidants, liquid fatty acid esters and hydrocarbons, emollients, emulsions, humectants, lubricants, preservatives, solvents, surfactants, acetate, acetone, Acetylated Lanolin Alcohol, Acrylates Copolymer, Acrylates/Octylpropenamide Copolymer, Alcohol SD-40, Algae/Seaweed Extract, Allantoin, Alpha Hydroxy Acid, Alpha Lipoic Acid, Alum, Ascorbic Acid, Ascorbyl Palmitate, Beeswax, Benzoyl Peroxide, Beta Hydroxy Acid, Boric Acid, Caffeine, Camphor, Carbomers (934, 940, 941, 980, 981), Carmine, Caviar (Roe Extract), Cellulose, Ceramides, Ceteareth, Cetyl Alcohol, Collagen, Cyclic Acid, Cyclomethicone, Dimethicone, EDTA, Elastin, Ellagic Acid, Ethyl Alcohol, Glycerin, Glycine, Glycogen, Glycolic Acid, Glycol Stearate, Grape Seed Extract, Green Tea Extract, Hyaluronic Acid, Hydroquinone, Isopropyl Alcohol, Isopropyl Isostearate, Isoproply Palmitate, Isostearic Acid, Kaolin (China Clay), Kojic Acid, Lactic Acid, Lanolin, Lecithin, L-Ergothioneine, Licorice Extract, Linoleic Acid, Lysine amino acid, Octyl Methoxycinnamate, Octyl Palmitate, Octyl Salicylate, Oxybenzone, PABA (Para-Aminobenzoic Acid), Panthenol, Parabens, Petrolatum, Polybutene, Poly Hydroxy Acid, Proline, Propylene Glycol, Resveratrol, Retinol, Retinyl Palmitate, Retinyl Palmitate Polypeptide, Rose Hips, Salicylic Acid, Silica, Silcone, Silk Powder, Silk Proteins, Sodium Bicarbonate, Sodium Borate, Sodium Hyaluronate, Sodium Laurel Sulfate, Sorbic Acid, Sorbitol, Stearic Acid, Sulfur, Titanium Dioxide, Triclosan, Tyrosine, Vitamin A, Vitamin B, Vitamin C, Vitamin D, Vitamin E, Witch Hazel and Xanthan Gum.

6. The method of claim 1, wherein the gaseous nitric oxide is applied under an air impermeable wound cover surrounding the skin.

7. The method of claim 1, wherein gaseous nitric oxide is applied at a concentration of about 20-1000 ppm.

8. The method of claim 1, further comprising pretreatment step of cleaning the skin.

9. The method of claim 1, wherein the gaseous nitric oxide is applied simultaneously with the cosmetic agent to the skin.

10. The method of claim 1, wherein the gaseous nitric oxide is applied before the applying of the cosmetic agent to the skin.

11. The method of claim 1, wherein the gaseous nitric oxide is applied after the applying of the cosmetic agent to the skin.

12. A method of promoting the healing of damaged tissue comprising:
applying to the damaged tissue gaseous nitric oxide; and
applying to the damaged tissue one or more growth factors.

13. The method of claim 12, wherein the damaged tissue is selected from the group consisting of muscle, ligament, tendon, skin, and bone.

14. The method of claim 12, wherein the damaged tissue is damaged by surgical incisions, trauma, and pathological processes.

15. The method of claim 12, wherein the one or more growth factors are selected from the group consisting of FGF, fibroblast growth factor; KGF, kerantinocyte growth factor; PDGF, platelet-derived growth factor; TGF, transforming growth factor, VEGF, vascular endothelial growth factor; EFG, epidermal growth factor; CTGF, connective tissue growth factor; and GMCSF, granulocyte-monocyte colony stimulating factor.

16. The method of claim 12, wherein the gaseous nitric oxide is applied under an air impermeable wound cover surrounding the damaged tissue.

17. The method of claim 12, wherein gaseous nitric oxide is applied at a concentration of about 20-1000 ppm.

18. The method of claim 12, further comprising pretreatment step of cleaning the damaged tissue.

19. The method of claim 12, wherein the gaseous nitric oxide is applied simultaneously with the cosmetic agent to the damaged tissue.

20. The method of claim 12, wherein the gaseous nitric oxide is applied before the applying of the one or more growth factors to the damaged tissue.

21. The method of claim 12, wherein the gaseous nitric oxide is applied after the applying of the one or more growth factors to the damaged tissue.

22. A method for cosmetically treating the skin comprising:
applying to the skin gaseous nitric oxide; and
applying to the skin one or more growth factors.

23. The method of claim 22, wherein the one or more growth factors are selected from the group consisting of FGF, fibroblast growth factor; KGF, kerantinocyte growth factor; PDGF, platelet-derived growth factor; TGF, transforming growth factor, VEGF, vascular endothelial growth factor; EFG, epidermal growth factor; CTGF, connective tissue growth factor; and GMCSF, granulocyte-monocyte colony stimulating factor.

24. The method of claim 22, wherein the skin is the face.

25. The method of claim 22, wherein the skin is the hands.

26. The method of claim 22, wherein the gaseous nitric oxide is applied under an air impermeable wound cover surrounding the skin.

27. The method of claim 22, wherein gaseous nitric oxide is applied at a concentration of about 20-1000 ppm.

28. The method of claim 22, further comprising pretreatment step of cleaning the skin.

29. The method of claim 22, wherein the gaseous nitric oxide is applied simultaneously with the cosmetic agent to the skin.

30. The method of claim 22, wherein the gaseous nitric oxide is applied before the applying of the one or more growth factors to the skin.

31. The method of claim 22, wherein the gaseous nitric oxide is applied after the applying of the one or more growth factors to the damaged tissue.

32. A method for cosmetically treating the skin comprising:
applying steam to the skin; and
applying gaseous nitric oxide to the skin.
